(19) United States
(12) Patent Application Publication
(10) Pub. No.: US 2005/0040065 A1
(43) Pub. Date: Feb. 24, 2005

(54) Medical procedure kit

(76) Inventor: Peter T. O'Heeron, Houston, TX (US)

Correspondence Address: Clarence E. Eriksen;JACKSON WALKER L.L.P., Suite 2100, 112 E. Pecan Street, San Antonio, TX, 78205, US

(21) Appl. No.: 10/646,675

(22) Filed: Aug. 22, 2003

(51) Int. Cl. A61L 15/00

(52) U.S. Cl. 206/438

(57) ABSTRACT

The present invention provides a medical kit having one or more obturators for use with a trocar. The obturator comprises a shaft having a distal or penetration end for insertion into a patient and a handling or proximal end for gripping by a surgeon. The penetration end of the shaft comprises a tip for cutting and/or separating tissue. The proximal end of the obturator having a handle member shaped to define a grasping portion having an orientation indicator thereon. The orientation indicator of the present invention providing the surgeon with the relative position of the tip of the obturator with respect to the shaft.

This patent application is a continuation-in-part of, and claims priority from a co-pending U.S. patent application entitled “Obturator Tip for a Trocar”, having a serial number of ______ and a filing date of Aug. 1, 2003; the specification, claims and drawings of which are hereby incorporated by reference herein.

### FIELD OF THE INVENTION

The present invention relates generally to surgical instruments and, more particularly, to an obturator kit for use in conjunction with one or more trocars for penetrating an anatomical cavity of a patient to provide communication with the cavity during a surgical procedure.

### BACKGROUND OF THE INVENTION

Endoscopic surgery is a significant method of performing surgical operations and has become the surgical procedure of choice due to its patient care advantages over “open surgery”. A particular type of endoscopic surgery is laparoscopic surgery. A significant advantage of laparoscopic surgery over open surgery is the decreased post-operative recovery time. In most instances, a patient is able to leave the hospital within hours after laparoscopic surgery has been performed, whereas with open surgery, a patient requires several days of hospital time to recover. Additionally, laparoscopic surgery achieves decreased incidents of post-operative abdominal adhesions, decreased post-operative pain, and enhanced cosmetic results.

Conventionally, a laparoscopic surgical procedure begins with the insufflation of the abdominal cavity with carbon dioxide. The introduction of this gas into the abdominal cavity lifts the abdominal wall away from the internal viscera. The abdominal wall is then pierced or penetrated with a device known as a trocar. A trocar includes a housing assembly, a cannula assembly attached to the housing assembly to form a bore through the trocar, and a piercing element called an obturator.

The obturator slides through an access port formed on the upper end of the housing assembly and through the bore of the trocar. After insertion of the trocar through the abdominal wall of the patient, the obturator is removed by the surgeon while leaving the cannula protruding through the abdominal wall. Thus, the cannula may be fixed and placed by using a fascia device, and laparoscopic instruments can be inserted through the cannula to view internal organs and perform surgical procedures.

Traditionally, the piercing tip of the obturator of a trocar has employed a sharp cutting blade to assist the surgeon in penetrating the abdominal wall. These obturators with cutting tips cut the tissue and muscle of the patient such that recovery time on the part of the patient is required. Moreover, since the cutting tips are sharp, costly safety shield mechanisms are employed which operate to cover the obturator tip a short time after the obturator passes through the abdominal wall of the patient.

Certain trocars, for example, as disclosed in U.S. Pat. Nos. 5,817,601 to Goodwin and 5,591,192 to Privitera utilize a pair of blunt-edged blades or tissue separators which are located on the tip of the trocar to facilitate the penetration or dissection of tissue. Both of these trocar assemblies referred to in U.S. Pat. Nos. 5,817,061, and 5,591,192 are manufactured and sold by Ethicon Endo-Surgery, Inc. It is believed that the trocars, as described in the '061 and '192 patents have encountered significant problems in that the tip of the obturator may be prone to failure. In particular, the tip of the trocar as disclosed in the '061 and '192 patents are believed to have experienced incidents of snapping off during the insertion of the trocar.

### SUMMARY OF THE INVENTION

Accordingly, the present invention provides a medical procedure kit comprising one or more obturators designed for use with a trocar. The obturators of the present invention having a distal end for insertion into the patient and a proximal end for gripping by the surgeon. The distal end and the proximal end being integrally connected by a shaft to form a single unit. The distal end of the obturator of the present invention comprises a tip designed to cut and/or separate tissue. The proximal end of the obturator of the present invention comprises at least one orientation indicator designed to provide the surgeon with the relative position of the obturator tip during use. In one embodiment, the obturators of the present invention are provided in ready-to-use condition in an attractive packaging arrangement.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a perspective view of the medical procedure kit of one embodiment of the present invention.

FIG. 2 is a side elevation view of the proximal end of the obturator of one embodiment of the present invention illustrating the orientation indicator.

FIG. 3 is a perspective view of one embodiment of the present invention illustrating the use of a single unit obturator in conjunction with a trocar.

### DETAILED DESCRIPTION OF THE INVENTION

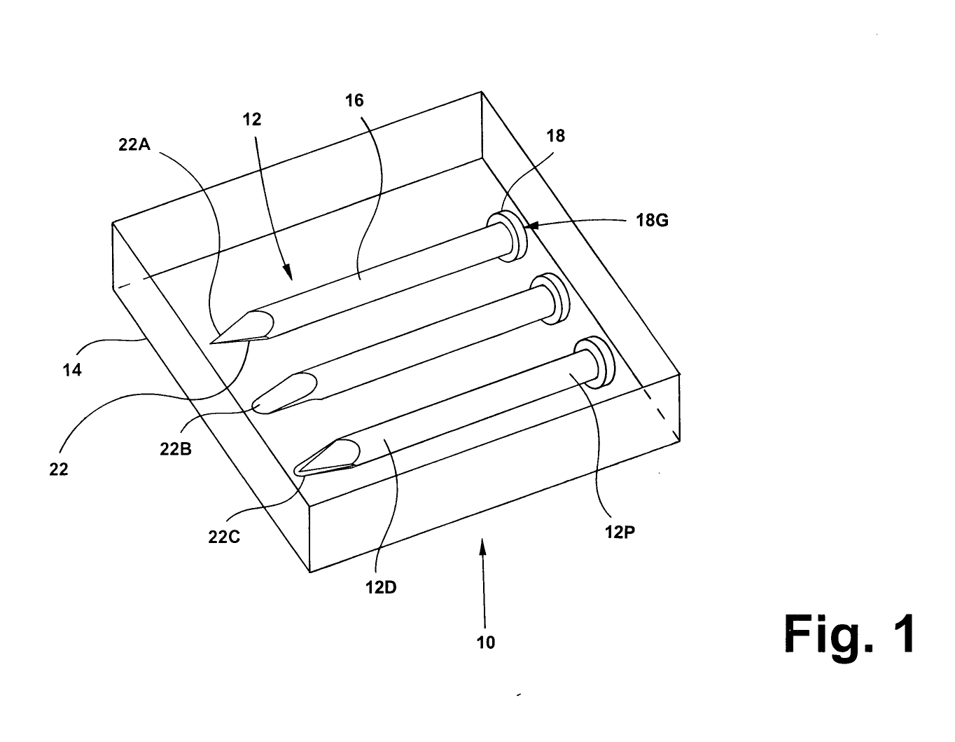

The present invention is herein described as a medical procedure kit. Referring to FIG. 1, the medical kit (10) of the present invention comprises at least one obturator (12) having a 20 distal or penetrating end (12D), a proximal or handling end (12P), and a shaft located there between. In one embodiment, both the distal end (12D) and the proximal end (12P) of the obturator (12) are fixed to the shaft (16) to form a single obturator unit.

The present invention provides for a monolithic obutrator (12) embodying a handled, shaft and geometric distal end for penetration of the fascia. This obutrator is unimpeded by the weight and complexity of a shielding mechanism. This makes the obutrator lighter, less expensive and no assembly required prior to shipment. While other disposable obturators have an internal or external shield, this oburator is designed to work without a shield or with a shield on the instrument cannula.

In one embodiment, the proximal end (12P) of the obturator is equipped with an arquate shaped handle member (18) having a grasping portion (18G) to facilitate insertion and maneuvering of the obturator (12) by the surgeon. The distal end (12D) of the obturator is equipped with a tip (22) capable of cutting and/or separating tissue.

The present invention provides a medical kit (10) having a plurality of obturators (12), each being designed for use with a correspondingly sized trocar (11), as discussed in greater detail below. Each obturator of the medical kit (10) may be equipped with a different tip (22) in order to provide the surgeon with the most useful array of instruments. For example, in one embodiment, the kit (10) of the present invention provides a first obturator having a cutting tip (22A), a second obturator having a substantially blunt tip (22B), and a third obturator having a tissue separating tip (22C). This feature of the present invention provides the surgeon with the most often utilized obturators in a convenient, east to use package (14).

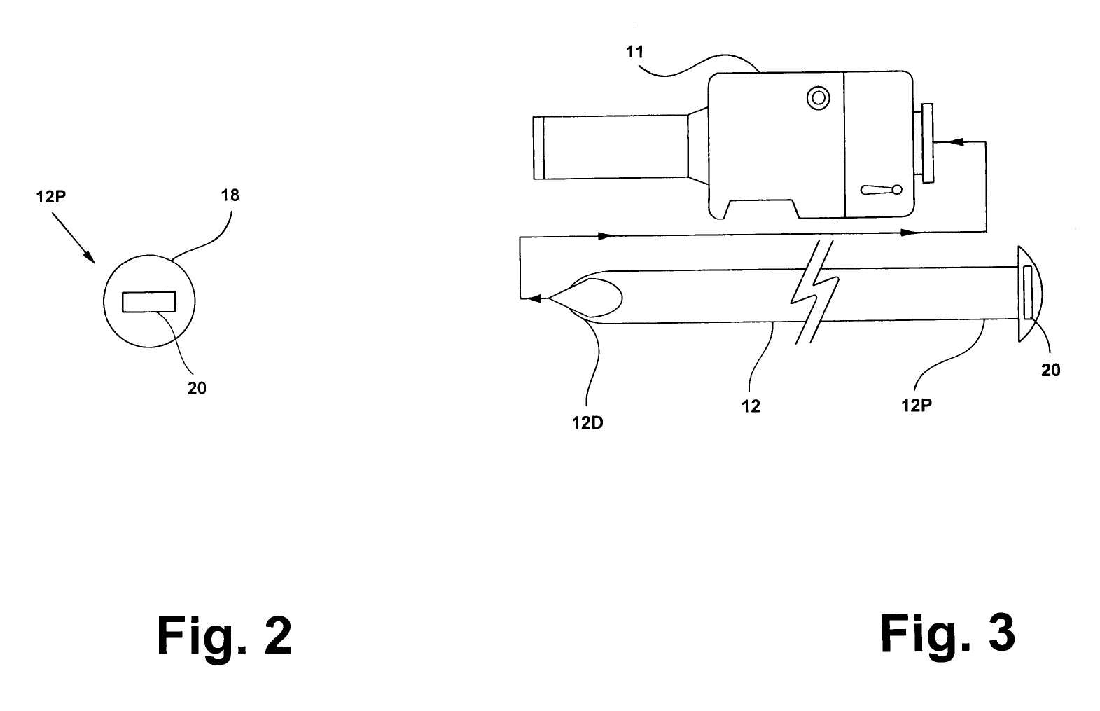

Referring to FIG. 2, the medical kit (10) of the present invention provides one or more orientation indicators (20) designed to assist the surgeon in ascertaining the relative position of the tip (22) of the obturator. In one embodiment, the handle member (18) of the obturator is equipped with one or more raised surfaces and/or indentions corresponding to the relative position of the tip (22) with respect to the shaft (16) of the trocar (12).

The use of raised surfaces and/or indentions upon the handle member (18) allows the surgeon to ascertain the rotational orientation of the tip without 1) removing his or her hand from the handle member, and 2) looking at the handle member. Specifically, the surgeon is able to feel the raised surfaces or indentions of the orientation indicator(s) and may continue working without interruption.

Referring to FIG. 3, the medical kit (10) of the present invention provides the surgeon with easy access to one or more obturators utilizing a host of tip designs. The single unit construction of the obturator(s) of the present invention allows the surgeon to remove and insert an obturator into the bore of the trocar (11). In one embodiment, graphical and/or textual information is placed upon each obturator to assist the surgeon or his/her assistant in ascertaining which obturator to utilize within a given trocar. For example, a color scheme may be applied to all or a portion of each obturator to correspond to color coating used by each trocar, e.g., green for 12 millimeter trocar, blue for 10 millimeter trocar, and red for 5 millimeter trocar.

The present invention is advantageous over known medical kits in that each obturator is disposable and does not require the removal/insertion of the tip (22). Specifically, obturators having multiple pieces, i.e., a separate handle, tip and/or shaft, require assembly prior to use by the surgeon. In some cases, manually attaching the tip (22) to the shaft (16) of the obturator (12) can be dangerous and lead to accidental injury or infection. Thus, the present invention not only provides the surgeon with convenient access to medical instruments, it also protects against human error prior to or during a medical procedure.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

1. A medical procedure kit comprising:
at least one shield-less obturator for use in one or more trocars, said obturator having a distal or penetration end, a proximal or handling end, and a shaft located between said distal end and said proximal ends; and
a package for enclosing said obturator.

2. The medical kit of claim 1, wherein said distal end and said proximal end are integral to said shaft such that each obturator is operable as a single unit.

3. The medical kit of claim 2, wherein each of said obturators is disposable.

4. The medical kit of claim 3, wherein said proximal end of each of said obturators comprises a handle member shaped to define a grasping portion.

5. The medical kit of claim 4, wherein at least one of said obturators has a distal end shaped to cut and/or separate tissue.

6. The medical kit of claim 5, wherein said handle member comprises at least one orientation indicator designed to provide the user with the relative position of said distal end of said obturator.

7. The medical kit of claim 6, wherein said orientation indicator comprises one or more raised surfaces and/or one or more indentations upon said grasping portion of said handle member.

8. The medical kit of claim 7, wherein said kit comprises three obturators, a first obturator having a cutting tip at its distal end, a second obturator having a substantially blunt tip at its distal end, and a third obturator having a tissue separating tip at its distal end.

9. The medical kit of claim 8, wherein at least one of said obturators is decorated with graphical and/or textual information for assisting the user in matching said obturator with a correspondingly sized trocar.

10. A medical procedure kit comprising:
at least three shield-less, disposable obturators for use in one or more trocars, each of said obturators having 1) a distal end shaped to cut and/or separate tissue, and 2) a proximal end shaped to define a grasping portion;
a shaft located between said distal end and said proximal end; and
a package for enclosing said obturators.

11. The medical kit of claim 10, wherein a first of said obturators has a cutting tip at its distal end, a second of said obturators has a substantially blunt tip at its distal end, and a third of said obturators has a tissue separating tip at its distal end.

12. The medical kit of claim 1 1, wherein said grasping portion of said proximal end of at least one of said obturators comprises at least one orientation indicator for providing the user with the relative position of said distal end of said obturator.

13. The medical kit of claim 12, wherein said orientation indicator comprises one or more raised surfaces and/or one or more indentions upon said grasping portion.

14. A medical procedure kit comprising:
at least three shield-less, disposable obturators for use in one or more trocars, each of said obturators having 1) a distal end shaped to cut and/or separate tissue, and 2) a proximal end shaped to define a grasping portion, said grasping portion having at least one orientation indicator for providing the relative position of said distal end, said orientation indicator comprising one or more raised surfaces and/or one or more indentions;
a shaft located between said distal end and said proximal end; and
a package for enclosing said obturators.
